(19) United States
(12) Patent Application Publication
(10) Pub. No.: US 2026/0090965 A1
(43) Pub. Date: Apr. 2, 2026

(54) DEPILATORY EMULSION CONTAINING PACLITAXEL AND PREPARATION METHOD THEREOF

(71) Applicant: Zhejiang Chinese Medical University, Hangzhou (CN)

(72) Inventor: Ming Xia, Hangzhou (CN)

(21) Appl. No.: 19/318,701

(22) Filed: Sep. 4, 2025

(30) Foreign Application Priority Data
Sep. 30, 2024 (CN) 2024113788581

(51) Int. Cl. A61K 8/49; A61K 8/06; A61K 8/19; A61K 8/34; A61K 8/36; A61K 8/37; A61K 8/92; A61Q 9/04

(52) U.S. Cl. CPC A61K 8/4973; A61K 8/06; A61K 8/19; A61K 8/342; A61K 8/345; A61K 8/361; A61K 8/375; A61K 8/498; A61K 8/4993; A61K 8/922; A61Q 9/04

(57) ABSTRACT

A depilatory emulsion containing paclitaxel is made from the following raw materials: 10% to 20% of oil, 8% to 12% of humectant, 4% to 6% of auxiliary emulsifier, 4% to 6% of emulsifier, 8% to 12% of paclitaxel solution, 0.5% to 1.0% of preservative, 0.01% to 0.05% of mineral, 0.05% to 0.10% of fragrance essential oil, and water for balance. This depilatory emulsion is designed to allow the paclitaxel and other active depilatory components to be absorbed by hair follicles while preventing absorption into dermis, thereby achieving advantages of an effective depilation, safety, absence of odor, reduced risk of skin allergy, and suitability for continuous use. A preparation method of this depilatory emulsion employs stepwise cooling and gradual emulsification to ensure that all components are fully dispersed and uniformly distributed, forming a stable emulsion system, which has advantages of simple steps and good emulsion stability.

### CROSS-REFERENCE TO RELATED APPLICATION

This application claims priority to Chinese Patent Application No. 202411378858.1, filed on Sep. 30, 2024, which is herein incorporated by reference in its entirety.

### TECHNICAL FIELD

The disclosure relates the technical field of special purpose cosmetics, and more particularly to a depilatory emulsion containing paclitaxel and a preparation method thereof.

### BACKGROUND

Human hair comes in a wide variety of types, and depilation (hair removal) primarily targets fine hairs distributed on the face, neck, torso, and limbs. Based on a depth of the depilation, common depilation methods can be classified into two major categories. The first category of depilation methods only removes visible hair shafts on a skin surface, including methods such as shaving and using chemical depilatories. The second category of depilation methods aims to completely remove not only the hair shafts but also hair bulbs within hair follicles, including methods such as plucking, waxing, sugaring, and laser depilation. However, both categories of depilation methods have significant drawbacks.

The first category of depilation methods is fast and convenient, but has a short duration of maintenance. The shaving, while convenient, can cause skin cuts if not performed properly and may lead to pseudofolliculitis, especially for those with sensitive skin. The chemical depilatories typically act on disulfide bonds in hair, using alkaline substances or thioglycolic acid and its salts to break down a hair structure, making the hair brittle and easy to remove by wiping. However, the chemical depilatories can irritate skin, causing allergic reactions and are not suitable for frequent use. Thiols, in addition to potentially causing allergies, may also produce an unpleasant odor. A patent with a Chinese patent publication No.CN115282080A discloses a depilation formulation containing enzymes that can gently break down keratin in the hair. However, long-term use of this formulation may damage stratum corneum of the skin, reduce skin resistance, and increase the risk of infection.

Although the second category of depilation methods provides longer-lasting results, it also causes greater damage to the skin. The plucking and the waxing can cause significant pain and are prone to causing local redness, folliculitis, and hair ingrown. The sugaring is only applicable to longer hairs and may cause skin allergies. Laser depilation varies in effectiveness from person to person, and its main adverse reactions include burns, scabbing, and pigmentation.

In the medical field, there is also a method that employs an eflornithine hydrochloride cream to slow down growth of facial hair in women. However, this cream has significant side effects, which may cause skin irritation, itching, redness, and swelling. It may also lead to increased skin pigmentation. Therefore, the eflornithine hydrochloride cream should be used cautiously under guidance of a physician.

Paclitaxel, a compound derived from Taxus chinensis through extraction and purification, can inhibit cell mitosis and only acts on cells in a mitotic phase, without affecting cells in interphase. Therefore, the paclitaxel is used as an anti-cancer drug. Paclitaxel is insoluble in water and cannot be absorbed into bloodstream through the skin or gastrointestinal tract, so it is usually administered intravenously in the form of an injection. After entering the bloodstream, the paclitaxel often causes severe allergic reactions in human body and can lead to a certain degree of bone marrow suppression, resulting in abnormal blood routine indicators. Therefore, Taxus chinensis is explicitly listed as a “Prohibited Item for Health Food” in the “Notice on Further Standardizing the Management of Health Food Ingredients” issued by the Ministry of Health.

A study on the application of Taxus chinensis extract in daily chemical products is recently published in Green Technology, 23(11), 3.128-130. After effective detoxification, active components such as volatile oils and flavonoids in the Taxus chinensis are essentially retained and can be used as raw materials or additives for the daily chemical products to produce functional daily chemical products. By utilizing the cytotoxicity of alkaloids in the Taxus chinensis and slightly retaining toxic alkaloids, local hair follicles can be stimulated, hair growth can be hindered, and hair loss can be promoted. This is used for the development of depilatory products. The “Regulations on the Supervision and Administration of Cosmetics” and the “Directory of Health Functions Allowed for Health Food Claims” issued at the end of 2020 recognize that the daily chemical products can make efficacy claims based on sufficient scientific evidence. Multiple studies prove the active effects of various components of the Taxus chinensis, showing that the application of the Taxus chinensis in the daily chemical products is reasonable and scientific. Based on extraction processes and detoxification technologies, an entire plant of the Taxus chinensis, including branches, leaves, and roots, can be fully utilized. Developing functional daily chemical products such as cosmetics and fragrances is a new way to achieve reasonable and efficient use of Taxus chinensis resources, with great commercial prospects and value. At the same time, it is also of great significance for guiding industrial production of Taxus chinensis functional daily chemical products.

Transdermal absorption of the cosmetics typically refers to a process by which active components in the cosmetics penetrate an outer layer of the skin and reach deeper skin tissues. Given that the skin surface is covered with a stratum corneum, which makes it difficult for these active components to penetrate. Therefore, current transdermal absorption techniques used in the cosmetics industry mainly focus on utilizing intercellular spaces, which account for approximately 30% of a total volume of the stratum corneum and provide a pathway for the active components to enter deeper skin layers, becoming a key route for the transdermal absorption. In contrast, the hair follicles, which occupy a very small proportion of a skin surface region and are isolated from the dermis by an epithelial sheath, are not primarily relied upon for absorption by the cosmetics currently on the market. Even when detoxified Taxus chinensis extract is used in the depilatory products, it is difficult to ensure that alkaloid components (such as paclitaxel) are confined to the hair follicles and do not penetrate into the dermis. Further research and improvement are still needed on this point.

### SUMMARY

In view of deficiencies of the related art, a first purpose of the disclosure is to provide a depilatory emulsion containing paclitaxel. The depilatory emulsion containing paclitaxel is designed to allow the paclitaxel and other active depilatory components to be absorbed by hair follicles while preventing absorption into dermis. The depilatory emulsion containing paclitaxel has advantages of an effective depilation, safety, absence of odor, reduced risk of skin allergy, and suitability for continuous use.

A second purpose of the disclosure is to provide a preparation method of the depilatory emulsion containing paclitaxel, which has advantages of simple steps and good emulsion stability.

To achieve the first purpose described above, the disclosure provides the following technical solution: a depilatory emulsion containing paclitaxel, made from the following raw materials by mass percent: 10% to 20% of oil, 8% to 12% of humectant, 4% to 6% of auxiliary emulsifier, 4% to 6% of emulsifier, 8% to 12% of paclitaxel solution, 0.5% to 1.0% of preservative, 0.01% to 0.05% of mineral, 0.05% to 0.10% of fragrance essential oil, and water for balance.

In an embodiment, the oil is one or a composition of safflower oil and wheat germ oil.

In an embodiment, the humectant is glycerin.

In an embodiment, the auxiliary emulsifier includes stearic acid and cetyl alcohol, and a mass ratio of the stearic acid to the cetyl alcohol is (1-3):1.

In an embodiment, the emulsifier includes monostearin, polysorbate 80 and sorbitan monostearate, and a mass ratio of the glyceryl monostearate, the polysorbate 80 and the sorbitan monostearate is (2-5):(2-3):1.

In an embodiment, a paclitaxel concentration in the paclitaxel solution is in a range of 0.15 milligrams per liter (mg/L) to 2.00 mg/L, and a solvent in the paclitaxel solution is ethanol with a volume fraction of 90% to 100% (also referred to as 90% to 100% v/v ethanol). The paclitaxel solution can be a pure solution obtained by dissolving purified paclitaxel directly in the 90% to 100% v/v ethanol, or it can be an extract obtained by extracting paclitaxel from roots, stems, leaves, and bark of Taxus chinensis using various methods, and the solvent in the extract is 90% to 100% v/v ethanol.

In an embodiment, the preservative includes kojic acid and parabens, and a mass ratio of the kojic acid to the parabens is in a range of (3-5):1.

In an embodiment, the mineral is one ore a composition of potassium hydroxide and potassium salts. The potassium salts are common forms including potassium chloride, potassium sulfate, and potassium carbonate.

In an embodiment, the fragrance essential oil is peppermint essential oil. A main component of the peppermint essential oil is menthol (also referred to as peppermint camphor), which is a colorless needle-like crystalline or granular substance and is a main component in Mentha canadensis and Mentha longifolia essential oils. The peppermint essential oil can be obtained by dissolving naturally purified or artificially synthesized menthol directly into commonly used cosmetic fragrances, with no limitation on fragrance types as long as it can dissolve menthol. Alternatively, the peppermint essential oil can also be extracted directly from peppermint plants.

To achieve the second purpose, the disclosure further provides the following technical solution: a preparation method of the depilatory emulsion containing paclitaxel, including the following steps:
S1, preparing the raw materials including the oil, the humectant, the auxiliary emulsifier, the emulsifier, the paclitaxel solution, the preservative, the mineral, the fragrance essential oil and the water according to a formulation ratio;
S2, heating the oil to 70° C. to 85° C., and adding the auxiliary emulsifier into the oil, followed by stirring to disperse, thereby obtaining an oil phrase;
S3, heating the water to above 95° C., and adding the humectant into the water, followed by stirring evenly to obtain a mixture; and adding the emulsifier into the mixture, followed by stirring to disperse, thereby obtaining an emulsion;
S4, controlling a temperature of the emulsion obtained in step S3 to 70° C. to 85° C., and slowly injecting the oil phrase obtained in step S2 into the emulsion, followed by stirring evenly to obtain a matrix; and
S5, slowly cooling the matrix obtained in step S4 to 45° C. to 55° C., and sequentially adding the paclitaxel solution, the preservative, the fragrance essential oil and the mineral into the matrix, followed by stirring to disperse evenly and cooling to below 40° C. to obtain the depilatory emulsion containing paclitaxel.

In summary, the disclosure may achieve the following beneficial effects.
1. Unlike mechanisms of action of traditional depilatory cosmetics, the depilatory emulsion containing paclitaxel of the disclosure ingeniously utilizes a natural growth cycle of hair. Since paclitaxel only acts on rapidly dividing cells, after applying the depilatory emulsion containing paclitaxel of the disclosure, hairs in an anagen/growth phase will cease growth. As the hairs in a catagen phase naturally shed, a number of the hairs on a skin surface gradually decreases, and an entire process is free of discomfort. Therefore, in terms of a mechanism of action of the depilatory emulsion containing paclitaxel, the disclosure effectively addresses issues that may arise from traditional depilatory methods, such as contact dermatitis, abnormal pigmentation, and follicular damage.
2. The depilatory emulsion containing paclitaxel of the disclosure combines dual functions of depilation and skin moisturizing, and a primary function of the depilatory emulsion containing paclitaxel is to remove hair. However, continuous use is required to maintain depilation effect. To this end, the depilatory emulsion containing paclitaxel incorporates components such as the glycerin and the kojic acid, which ensure that the depilatory emulsion containing paclitaxel not only maintains its depilation efficacy but also exerts a moisturizing effect.
3. Due to barrier effect of stratum corneum on the skin surface against lipophilic compounds such as paclitaxel, and considering that sebaceous glands typically open into hair follicles which contain free fats, the depilatory emulsion containing paclitaxel of the disclosure, through synergistic action of the free fats and emulsifiers, enables paclitaxel to penetrate along interstices of the hair follicles to reach hair matrix cells, thereby inhibiting hair growth. Meanwhile, due to obstruction by epithelial cells lining inner walls of the hair follicles, paclitaxel does not permeate into dermis or bloodstream, thus avoiding common allergic reactions associated with the injection of paclitaxel.
4. Paclitaxel is a low-toxicity and highly effective anticancer drug, which primarily functions by stabilizing microtubules and disrupting the normal progression of mitosis. As a result, it selectively acts on the hair matrix cells in a rapid growth phase, while being harmless to those in a resting phase. This selective action endows the depilation effect of the disclosure with reversibility. That's to say, after discontinuation of the depilatory emulsion containing paclitaxel of the disclosure for 1 month to 2 months, the hair matrix cells in the resting phase will be reactivated, and hair will gradually resume growth.
5. Common skin permeation enhancers primarily focus on improving the permeability of intercellular spaces in the stratum corneum. However, the depilatory emulsion containing paclitaxel of the disclosure needs to both promote the entry of paclitaxel into the hair follicles and prevent its entry into the bloodstream. To this end, the disclosure employs a combination strategy of menthol and the humectant. Specifically, the humectant constructs a hydrophilic barrier in the stratum corneum of the skin to prevent the permeation of paclitaxel into the dermis, while the menthol, leveraging its pore-dilating properties, facilitates the entry of paclitaxel into the interior of the hair follicles through pores.
6. The depilatory emulsion containing paclitaxel of the disclosure takes advantage of enhanced potassium uptake capacity of rapidly dividing hair matrix cells. In addition, the transmembrane transport of paclitaxel into the hair matrix cells requires energy provided by sodium-potassium ion pumps. Therefore, potassium-containing minerals added in the disclosure facilitate further absorption of paclitaxel by the hair matrix cells within the hair follicles.
7. To ensure that the depilatory emulsion containing paclitaxel of the disclosure has excellent coating properties and does not produce a greasy feel upon application, it is formulated as an oil-in-water (O/W) emulsion. During a preparation process, a method of the disclosure employs a stepwise cooling and gradual emulsification approach to ensure that all components are fully dispersed and uniformly distributed, thereby forming a stable emulsion system. The resulting emulsion containing paclitaxel is of stable quality and does not exhibit phase separation.

### BRIEF DESCRIPTION OF DRAWINGS

FIG. 1 illustrates a schematic diagram of an appearance of a depilatory emulsion containing paclitaxel according to an embodiment 1 of the disclosure.

FIG. 2 illustrates a schematic diagram of a stability test of the depilatory emulsion containing paclitaxel according to the embodiment 1 of the disclosure.

FIG. 3 illustrates a schematic diagram of total colony count detection results of a depilatory emulsion containing paclitaxel according to an embodiment 2 of the disclosure.

FIG. 4 illustrates a chromatogram of the depilatory emulsion containing paclitaxel according to the embodiment 2 of the disclosure.

FIG. 5 illustrates a schematic diagram of an external auricle of a rabbit treated with the depilatory emulsion containing paclitaxel according to the embodiment 2 of the disclosure.

FIG. 6 illustrates a schematic diagram of hair shedding conditions after using the depilatory emulsion containing paclitaxel according to the embodiment 1 of the disclosure.

### DETAILED DESCRIPTION OF EMBODIMENTS

To make technical means, creative features, purposes and functions of the disclosure clearer and easier to understand, the following will further elaborate on the disclosure in conjunction with attached drawings and specific embodiments.

### Preparation Example 1

A certain amount of paclitaxel (obtained from Chengdu Must Bio-Technology Co., Ltd.) is weighed accurately. The paclitaxel is then dissolved in a 90% (v/v) ethanol solution (i.e., an ethanol solution with a volume fraction of 90%) and diluted to a paclitaxel concentration of 0.5 mg/L, yielding a paclitaxel/ethanol solution.

### Preparation Example 2

Bark of Taxus chinensis is ground to a particle size of 100 mesh and added to an extraction vessel of a supercritical carbon dioxide extraction apparatus. Then ethanol with a water content of 10% v/v (i.e., a 90% v/v ethanol solution) is added to the extraction vessel as a co-solvent and mixed evenly to obtain a mixture. A temperature of the mixture is raised to 35° C., and a pressure of the mixture is increased to 35 megapascals (MPa). Extraction is performed on the mixture for 120 minutes. Subsequently, carbon dioxide is depressurized and released in a separation vessel of the supercritical carbon dioxide extraction apparatus, yielding an extract containing paclitaxel. Anhydrous ethanol is added to the extract containing paclitaxel to dilute a paclitaxel concentration to 0.15 mg/L, followed by filtering with a 0.45 micrometers (μm) microporous filter membrane to obtain a paclitaxel extract solution.

### Embodiment 1

The disclosure provides a depilatory emulsion containing paclitaxel, which is made from the following raw materials by mass percent: 13% of oil, 9% of humectant, 6% of auxiliary emulsifier, 4.4% of emulsifier, 8% of paclitaxel solution, 1% of preservative, 0.02% of mineral, 0.05% of fragrance essential oil, and water for balance. Specifically, the oil is safflower oil. The humectant is glycerin. The auxiliary emulsifier includes stearic acid and cetyl alcohol, and a mass ratio of the stearic acid to the cetyl alcohol is 2:1. The emulsifier includes monostearin, polysorbate 80 and sorbitan monostearate, and a mass ratio of the glyceryl monostearate, the polysorbate 80 and the sorbitan monostearate is 2.86:2.43:1. The paclitaxel solution is the paclitaxel/ethanol solution prepared in the preparation example 1. The preservative includes kojic acid and parabens, and a mass ratio of the kojic acid to the parabens is 4:1. The mineral is potassium hydroxide. The fragrance essential oil is peppermint essential oil prepared by dissolving menthol directly in a fragrance base.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 80° C., and the auxiliary emulsifier is added into the oil, followed by stirring to disperse, thereby obtaining an oil phrase.

In step S3, the water is heated to 95° C., and adding the humectant into the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 80° C., and the oil phrase obtained in the step S2 is slowly injected into the emulsion, followed by stirring evenly to obtain a matrix.

In step S5, the matrix obtained in the step S4 is slowly cooled to 45° C., and the paclitaxel solution, the preservative, the fragrance essential oil and the mineral are sequentially added into the matrix, followed by stirring to disperse evenly and cooling to below 40° C. to obtain the depilatory emulsion containing paclitaxel.

### Embodiment 2

The disclosure provides a depilatory emulsion containing paclitaxel, which is made from the following raw materials by mass percent: 15% of oil, 10% of humectant, 4% of auxiliary emulsifier, 6% of emulsifier, 10% of paclitaxel solution, 0.8% of preservative, 0.05% of mineral, 0.1% of fragrance essential oil, and water for balance. Specifically, the oil is wheat germ oil. The humectant is glycerin. The auxiliary emulsifier includes stearic acid and cetyl alcohol, and a mass ratio of the stearic acid to the cetyl alcohol is 3:1. The emulsifier includes monostearin, polysorbate 80 and sorbitan monostearate, and a mass ratio of the glyceryl monostearate, the polysorbate 80 and the sorbitan monostearate is 3:2:1. The paclitaxel solution is the paclitaxel extract solution prepared in the preparation example 2. The preservative includes kojic acid and parabens, and a mass ratio of the kojic acid to the parabens is 3:1. The mineral is potassium chloride. The fragrance essential oil is peppermint essential oil extracted from peppermint plants.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 85° C., and the auxiliary emulsifier is added into the oil, followed by stirring to disperse, thereby obtaining an oil phrase.

In step S3, the water is heated to 100° C., and adding the humectant into the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 85° C., and the oil phrase obtained in the step S2 is slowly injected into the emulsion, followed by stirring evenly to obtain a matrix.

In step S5, the matrix obtained in the step S4 is slowly cooled to 55° C., and the paclitaxel solution, the preservative, the fragrance essential oil and the mineral are sequentially added into the matrix, followed by stirring to disperse evenly and cooling to below 40° C. to obtain the depilatory emulsion containing paclitaxel.

### Embodiment 3

The disclosure provides a depilatory emulsion containing paclitaxel, which is made from the following raw materials by mass percent: 10% of oil, 8% of humectant, 4% of auxiliary emulsifier, 4% of emulsifier, 8.5% of paclitaxel solution, 0.5% of preservative, 0.01% of mineral, 0.05% of fragrance essential oil, and water for balance. Specifically, the oil is safflower oil. The humectant is glycerin. The auxiliary emulsifier includes stearic acid and cetyl alcohol, and a mass ratio of the stearic acid to the cetyl alcohol is 1:1. The emulsifier includes monostearin, polysorbate 80 and sorbitan monostearate, and a mass ratio of the glyceryl monostearate, the polysorbate 80 and the sorbitan monostearate is 2:2:1. The paclitaxel solution is the paclitaxel/ethanol solution prepared in the preparation example 1. The preservative includes kojic acid and parabens, and a mass ratio of the kojic acid to the parabens is 3:1. The mineral is potassium hydroxide. The fragrance essential oil is peppermint essential oil prepared by dissolving menthol directly in a fragrance base.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 70° C., and the auxiliary emulsifier is added into the oil, followed by stirring to disperse, thereby obtaining an oil phrase.

In step S3, the water is heated to 95° C., and adding the humectant into the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 70° C., and the oil phrase obtained in the step S2 is slowly injected into the emulsion, followed by stirring evenly to obtain a matrix.

In step S5, the matrix obtained in the step S4 is slowly cooled to 46° C., and the paclitaxel solution, the preservative, the fragrance essential oil and the mineral are sequentially added into the matrix, followed by stirring to disperse evenly and cooling to below 38° C. to obtain the depilatory emulsion containing paclitaxel.

### Embodiment 4

The disclosure provides a depilatory emulsion containing paclitaxel, which is made from the following raw materials by mass percent: 12% of oil, 9% of humectant, 4% of auxiliary emulsifier, 4% of emulsifier, 9% of paclitaxel solution, 0.6% of preservative, 0.02% of mineral, 0.06% of fragrance essential oil, and water for balance. Specifically, the oil is safflower oil. The humectant is glycerin. The auxiliary emulsifier includes stearic acid and cetyl alcohol, and a mass ratio of the stearic acid to the cetyl alcohol is 1.5:1. The emulsifier includes monostearin, polysorbate 80 and sorbitan monostearate, and a mass ratio of the glyceryl monostearate, the polysorbate 80 and the sorbitan monostearate is 2.5:2.5:1. The paclitaxel solution is the paclitaxel/ethanol solution prepared in the preparation example 1. The preservative includes kojic acid and parabens, and a mass ratio of the kojic acid to the parabens is 3.5:1. The mineral is potassium hydroxide. The fragrance essential oil is peppermint essential oil prepared by dissolving menthol directly in a fragrance base.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 72° C., and the auxiliary emulsifier is added into the oil, followed by stirring to disperse, thereby obtaining an oil phrase.

In step S3, the water is heated to 100° C., and adding the humectant to the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 72° C., and the oil phrase obtained in the step S2 is slowly injected into the emulsion, followed by stirring evenly to obtain a matrix.

In step S5, the matrix obtained in the step S4 is slowly cooled to 48° C., and the paclitaxel solution, the preservative, the fragrance essential oil and the mineral are sequentially added into the matrix, followed by stirring to disperse evenly and cooling to below 35° C. to obtain the depilatory emulsion containing paclitaxel.

### Embodiment 5

The disclosure provides a depilatory emulsion containing paclitaxel, which is made from the following raw materials by mass percent: 14% of oil, 10% of humectant, 5% of auxiliary emulsifier, 5% of emulsifier, 9.5% of paclitaxel solution, 0.7% of preservative, 0.03% of mineral, 0.07% of fragrance essential oil, and water for balance. Specifically, the oil is safflower oil. The humectant is glycerin. The auxiliary emulsifier includes stearic acid and cetyl alcohol, and a mass ratio of the stearic acid to the cetyl alcohol is 1:1. The emulsifier includes monostearin, polysorbate 80 and sorbitan monostearate, and a mass ratio of the glyceryl monostearate, the polysorbate 80 and the sorbitan monostearate is 3.5:3:1. The paclitaxel solution is the paclitaxel/ethanol solution prepared in the preparation example 1. The preservative includes kojic acid and parabens, and a mass ratio of the kojic acid to the parabens is 4:1. The mineral is potassium carbonate. The fragrance essential oil is peppermint essential oil prepared by dissolving menthol directly in a fragrance base.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 74° C., and the auxiliary emulsifier is added into the oil, followed by stirring to disperse, thereby obtaining an oil phrase.

In step S3, the water is heated to 95° C., and adding the humectant into the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 74° C., and the oil phrase obtained in the step S2 is slowly injected into the emulsion, followed by stirring evenly to obtain a matrix.

In step S5, the matrix obtained in the step S4 is slowly cooled to 50° C., and the paclitaxel solution, the preservative, the fragrance essential oil and the mineral are sequentially added into the matrix, followed by stirring to disperse evenly and cooling to below 32° C. to obtain the depilatory emulsion containing paclitaxel.

### Embodiment 6

The disclosure provides a depilatory emulsion containing paclitaxel, which is made from the following raw materials by mass percent: 16% of oil, 10% of humectant, 5% of auxiliary emulsifier, 5% of emulsifier, 10.5% of paclitaxel solution, 0.8% of preservative, 0.04% of mineral, 0.08% of fragrance essential oil, and water for balance. Specifically, the oil is safflower oil. The humectant is glycerin. The auxiliary emulsifier includes stearic acid and cetyl alcohol, and a mass ratio of the stearic acid to the cetyl alcohol is 1.5:1. The emulsifier includes monostearin, polysorbate 80 and sorbitan monostearate, and a mass ratio of the glyceryl monostearate, the polysorbate 80 and the sorbitan monostearate is 4:2:1. The paclitaxel solution is the paclitaxel/ethanol solution prepared in the preparation example 1. The preservative includes kojic acid and parabens, and a mass ratio of the kojic acid to the parabens is 4.5:1. The mineral is potassium hydroxide. The fragrance essential oil is peppermint essential oil prepared by dissolving menthol directly in a fragrance base.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 78° C., and the auxiliary emulsifier is added into the oil, followed by stirring to disperse, thereby obtaining an oil phrase.

In step S3, the water is heated to 100° C., and adding the humectant into the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 78° C., and the oil phrase obtained in the step S2 is slowly injected into the emulsion, followed by stirring evenly to obtain a matrix.

In step S5, the matrix obtained in the step S4 is slowly cooled to 50° C., and the paclitaxel solution, the preservative, the fragrance essential oil and the mineral are sequentially added into the matrix, followed by stirring to disperse evenly and cooling to below 30° C. to obtain the depilatory emulsion containing paclitaxel.

### Embodiment 7

The disclosure provides a depilatory emulsion containing paclitaxel, which is made from the following raw materials: 18% of oil, 11% of humectant, 6% of auxiliary emulsifier, 6% of emulsifier, 11% of paclitaxel solution, 0.9% of preservative, 0.04% of mineral, 0.09% of fragrance essential oil, and water for balance. Specifically, the oil is safflower oil. The humectant is glycerin. The auxiliary emulsifier includes stearic acid and cetyl alcohol, and a mass ratio of the stearic acid to the cetyl alcohol is 1:1. The emulsifier includes monostearin, polysorbate 80 and sorbitan monostearate, and a mass ratio of the glyceryl monostearate, the polysorbate 80 and the sorbitan monostearate is 4.5:2.5:1. The paclitaxel solution is the paclitaxel/ethanol solution prepared in the preparation example 1. The preservative includes kojic acid and parabens, and a mass ratio of the kojic acid to the parabens is 5:1. The mineral is potassium hydroxide. The fragrance essential oil is peppermint essential oil prepared by dissolving menthol directly in a fragrance base.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 82° C., and the auxiliary emulsifier is added into the oil, followed by stirring to disperse, thereby obtaining an oil phrase.

In step S3, the water is heated to 95° C., and adding the humectant into the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 82° C., and the oil phrase obtained in the step S2 is slowly injected into the emulsion, followed by stirring evenly to obtain a matrix.

In step S5, the matrix obtained in the step S4 is slowly cooled to 52° C., and the paclitaxel solution, the preservative, the fragrance essential oil and the mineral are sequentially added into the matrix, followed by stirring to disperse evenly and cooling to below 40° C. to obtain the depilatory emulsion containing paclitaxel.

### Embodiment 8

The disclosure provides a depilatory emulsion containing paclitaxel, which is made from the following raw materials by mass percent: 20% of oil, 12% of humectant, 6% of auxiliary emulsifier, 6% of emulsifier, 12% of paclitaxel solution, 1% of preservative, 0.05% of mineral, 0.1% of fragrance essential oil, and water for balance. Specifically, the oil is safflower oil. The humectant is glycerin. The auxiliary emulsifier includes stearic acid and cetyl alcohol, and a mass ratio of the stearic acid to the cetyl alcohol is 3:1. The emulsifier includes monostearin, polysorbate 80 and sorbitan monostearate, and a mass ratio of the glyceryl monostearate, the polysorbate 80 and the sorbitan monostearate is 5:3:1. The paclitaxel solution is the paclitaxel/ethanol solution prepared in the preparation example 1. The preservative includes kojic acid and parabens, and a mass ratio of the kojic acid to the parabens is 5:1. The mineral is potassium hydroxide. The fragrance essential oil is peppermint essential oil prepared by dissolving menthol directly in a fragrance base.

The disclosure further provides a preparation method of the depilatory emulsion containing paclitaxel, including the following steps S1 through S5.

In step S1, the raw materials including the oil, the humectant, the auxiliary emulsifier, the emulsifier, the paclitaxel solution, the preservative, the mineral, the fragrance essential oil and the water are prepared according to a formulation ratio.

In step S2, the oil is heated to 84° C., and the auxiliary emulsifier is added into the oil, followed by stirring to disperse, thereby obtaining an oil phrase.

In step S3, the water is heated to 100° C., and the humectant is added into the water, followed by stirring evenly to obtain a mixture. Then the emulsifier is added into the mixture, followed by stirring to disperse, thereby obtaining an emulsion.

In step S4, a temperature of the emulsion obtained in the step S3 is controlled at 84° C., and the oil phrase obtained in the step S2 is slowly injected into the emulsion, followed by stirring evenly to obtain a matrix.

In step S5, the matrix obtained in the step S4 is slowly cooled to 54° C., and the paclitaxel solution, the preservative, the fragrance essential oil and the mineral are sequentially added into the matrix, followed by stirring to disperse evenly and cooling to below 40° C. to obtain the depilatory emulsion containing paclitaxel.

### Test Example 1: PH Value Measurement

The depilatory emulsion containing paclitaxel prepared in the embodiment 2 is subjected to pH value measurement according to a method specified in GB/T13531.1-2008. One part of the depilatory emulsion containing paclitaxel is weighed and added with nine parts of deionized water to obtain a mixture. The mixture is stirred evenly at 40° C. and then cooled to 25° C. A pH value of the mixture is measured by using a pH meter with an accuracy of 0.1. The above test is repeated five times, and an average value of the pH values for five times is calculated.

Measurement results indicate a pH value of 7.0±0.2, which falls within an acceptable range for cosmetic pH values as stipulated by national standards.

### Test Example 2: Stability Test

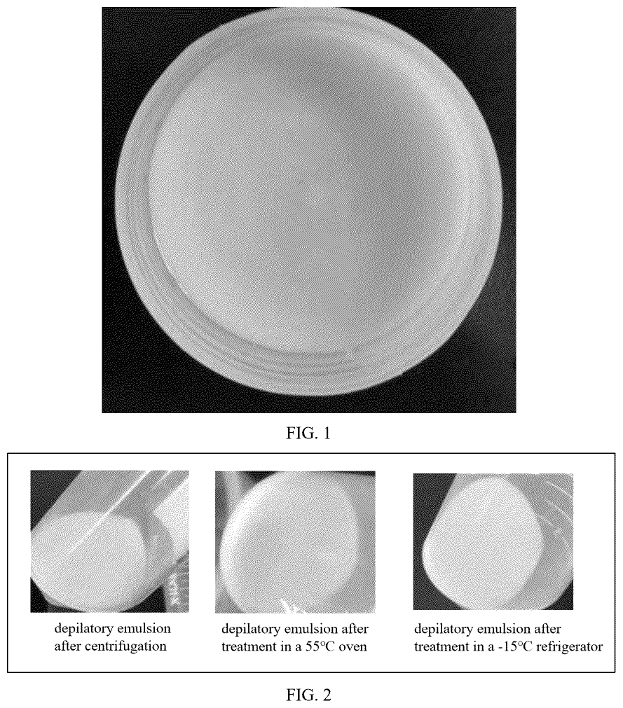

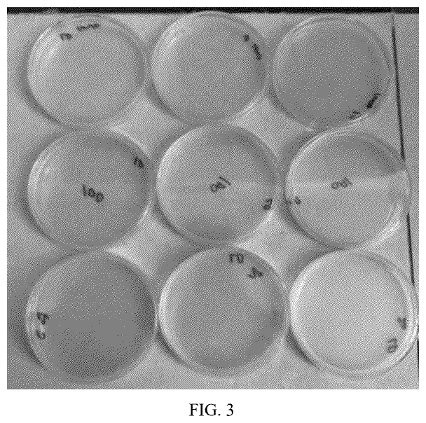

After allowing the depilatory emulsion containing paclitaxel prepared in the embodiment 1 to stand at room temperature for one week, 1.5 grams (g) of the depilatory emulsion containing paclitaxel is placed in a centrifuge tube and centrifuged at a speed of 4,000 revolutions per minute (rpm) for 30 minutes. The presence or absence of phase separation and the uniformity of color are observed. The depilatory emulsion containing paclitaxel is separately placed in an oven at 55° C. and a refrigerator at −15° C. for 24 hours to observe any liquefaction, hardening, or roughening, and results are shown in FIG. 2.

It can be seen from FIG. 2 that under extreme conditions set in the test, the depilatory emulsion containing paclitaxel has no oil emulsion stratification, liquefaction and hardening, indicating that this product (i.e., the depilatory emulsion containing paclitaxel) is stable in nature.

### Test Example 3: Total Colony Count Detection

The depilatory emulsion containing paclitaxel prepared in the embodiment 2 is subjected to total colony count detection according to a method specified in GB/T7918.1-1987. 10 g of the depilatory emulsion containing paclitaxel is weighed and placed into an Erlenmeyer flask (also referred to as conical flask), and 90 milliliters (mL) of sterile physiological saline is added into the Erlenmeyer flask to obtain a mixture. After thorough mixing by shaking, the mixture is allowed to stand in a water bath at 30-32° C. for 15 minutes to obtain a 1:10 dilution. Further dilutions are made by using sterile physiological saline to achieve 1:10, 1:100, and 1:1000 dilutions. The diluted solutions are then dispensed into sterile Petri dishes, with three dishes used for each dilution factor and 2 mL of diluted solution added to each dish.

A nutrient agar medium containing lecithin and polysorbate 80 (TWEEN® 80) is prepared following a method specified in GB/T7918.2-1987, after sterilized and cooled to 45-50° C., the nutrient agar medium containing lecithin and polysorbate 80 is poured into the dishes containing the diluted solutions, approximately 15 mL per dish. A blank control culture dish is also prepared. The dishes are rotated to ensure thorough mixing of the sample with the agar medium. After the agar is solidified, the dishes are inverted and incubated in an incubator at 37° C. for 48 hours. Following a 48-hour incubation period, colonies are visually counted, and an average number of colonies growing on dishes at a same dilution is calculated. Results are as shown in FIG. 3.

It can be seen from FIG. 3, the total bacterial count is less than 1000 colony forming units per gram (CFU/g), which complies with requirements of GB7916-1987.

### Test Example 4: Paclitaxel Content Detection

10 g of the depilatory emulsion containing paclitaxel prepared in the embodiment 2 is accurately weighed. 30 mL of methanol is added to the depilatory emulsion containing paclitaxel to obtain a mixture. The mixture is sonicated for 30 minutes to ensure homogeneity, and then is filtered and transferred to a 50 mL volumetric flask. Methanol is used to make up to a final volume, thereby preparing a sample solution. A standard curve for paclitaxel content in the depilatory emulsion containing paclitaxel is constructed following a method in the literature “Zhou Xiuling, Peng Quancai, Determination of paclitaxel content in branches and leaves of Taxus by high-performance liquid chromatography [J]. Shi Zhen Guo Yi Guo Yao, 2008, (06): 1434-1436”.

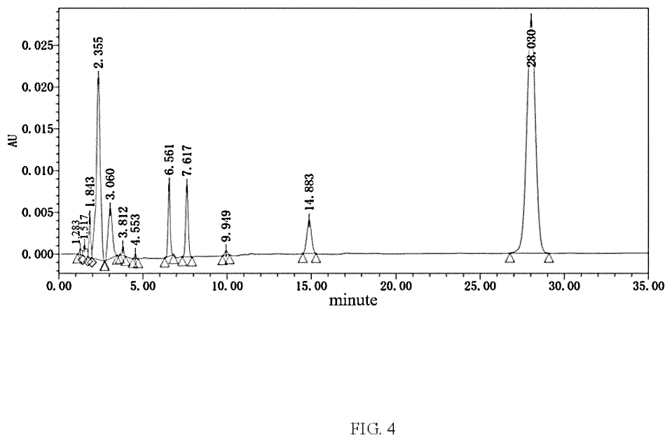

The paclitaxel content in the depilatory emulsion containing paclitaxel is 0.014 mg/L after detection. The chromatogram of the sample solution is shown in FIG. 4, where a peak at 28.030 minutes is identified as paclitaxel.

### Test Example 5: Animal Test

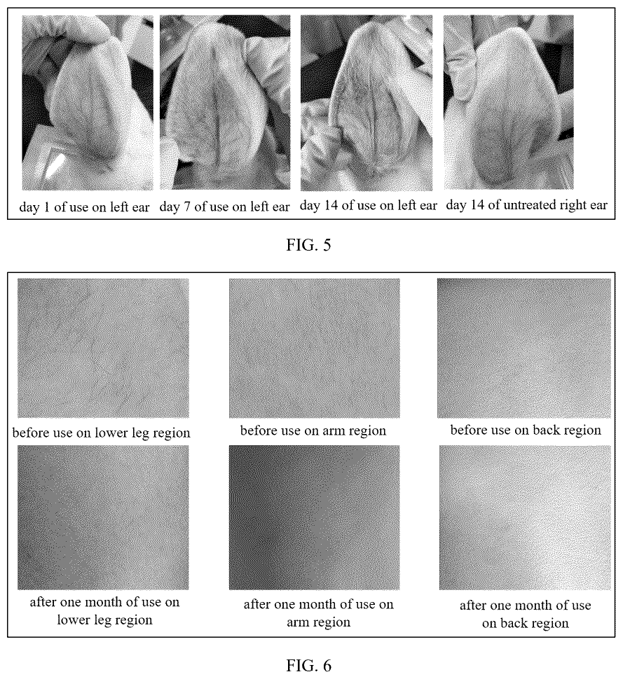

Rabbits weighing 2.5±0.2 kg are selected. The depilatory emulsion containing paclitaxel prepared in the embodiment 2 is applied to an external auricle of a left ear of each rabbit, approximately 0.05 mL per application, twice daily. A helix position of the left ear of each rabbit is not treated, and a right ear of each rabbit is left untreated, serving as a control. The rabbits are fed normally. On days 1, 7, and 14, the application sites are gently wiped with a damp towel, and photographs are taken after the surface had dried. On day 11, 2 hours after applying the depilatory emulsion containing paclitaxel, venous blood is drawn from the ear, and the paclitaxel content in the rabbit plasma is detected by using a method specified in a literature “Zhang Tianying, Guo Yuzhi, Zhang Dawei, et al., Validation of HPLC Method for Determining Paclitaxel Concentrations in Human Plasma [J]. Heilongjiang Medical Journal of Pharmaceutical Sciences, 2013, (2): 54-55”. A blood routine examination is also performed by using a hematological analyzer, and results are shown in FIG. 5.

It can be seen from FIG. 5 that there is a significant reduction in hair on the external auricle of the left ear of each rabbit treated with the depilatory emulsion containing paclitaxel, while no change in hair quantity is observed on the untreated right ear. Since rabbit hair is continuously renewed, significant depilation effects can be observed within 7 days. If paclitaxel enters the bloodstream, it could potentially impair bone marrow hematopoietic function. However, the blood routine examination shows no significant abnormalities in a number of various blood cells of each tested rabbits, and paclitaxel is not detected in the blood, indicating that an action of paclitaxel is limited to the surface and did not enter the bloodstream.

### Test Example 6: Allergenicity and Efficacy Verification Test

10 volunteers with abundant body hair are recruited. Arms, lower legs, and backs are designated as depilation test regions. A sufficient amount of the depilatory emulsion containing paclitaxel prepared in the embodiment 1 is applied twice daily, with moderate massage and rubbing. A test period lasts for one month. The efficacy of depilation is verified by observing the hair shedding in the test regions before and after the treatment. During a test period, the 10 volunteers record any adverse skin reactions in the depilation regions, including but not limited to erythema, itching, pigmentation abnormalities, contact dermatitis, and other symptoms. Results are shown in FIG. 6.

It can be seen from FIG. 6 that all of the 10 volunteers show good depilation effects, with a depilation rate of 90% at the application sites, no dark spots appear, and no allergic symptoms are observed.

Finally, it should be noted that the embodiments described above are provided merely to illustrate the technical solutions of the disclosure and are not intended to be limiting. Although the disclosure has been described in detail with reference to the specific embodiments, those skilled in the art should understand that modifications or equivalent substitutions to the technical solutions of the disclosure can be made without departing from the spirit and scope of the disclosure. All such modifications and equivalent substitutions are intended to be covered by the appended claims of the disclosure.

What is claimed is:

1. A depilatory emulsion containing paclitaxel, made from the following raw materials by mass percent:
10% to 20% of oil, 8% to 12% of humectant, 4% to 6% of auxiliary emulsifier, 4% to 6% of emulsifier, 8% to 12% of paclitaxel solution, 0.5% to 1.0% of preservative, 0.01% to 0.05% of mineral, 0.05% to 0.10% of fragrance essential oil, and water for balance.

2. The depilatory emulsion containing paclitaxel as claimed in claim 1, wherein the oil is one or a composition of safflower oil and wheat germ oil.

3. The depilatory emulsion containing paclitaxel as claimed in claim 1, wherein the humectant is glycerin.

4. The depilatory emulsion containing paclitaxel as claimed in claim 1, wherein the auxiliary emulsifier comprises stearic acid and cetyl alcohol, and a mass ratio of the stearic acid to the cetyl alcohol is (1-3):1.

5. The depilatory emulsion containing paclitaxel as claimed in claim 1, wherein the emulsifier comprises monostearin, polysorbate 80 and sorbitan monostearate, and a mass ratio of the glyceryl monostearate, the polysorbate 80 and the sorbitan monostearate is (2-5):(2-3):1.

6. The depilatory emulsion containing paclitaxel as claimed in claim 1, wherein a paclitaxel concentration in the paclitaxel solution is in a range of 0.15 milligrams per liter (mg/L) to 2.00 mg/L, and a solvent in the paclitaxel solution is ethanol with a volume fraction of 90% to 100%.

7. The depilatory emulsion containing paclitaxel as claimed in claim 1, wherein the preservative comprises kojic acid and parabens, and a mass ratio of the kojic acid to the parabens is in a range of (3-5):1.

8. The depilatory emulsion containing paclitaxel as claimed in claim 1, wherein the mineral is one or a composition of potassium hydroxide and potassium salts.

9. The depilatory emulsion containing paclitaxel as claimed in claim 1, wherein the fragrance essential oil is peppermint essential oil.

10. A preparation method of the depilatory emulsion containing paclitaxel as claimed in claim 1, comprising the following steps:
S1, preparing the raw materials comprising the oil, the humectant, the auxiliary emulsifier, the emulsifier, the paclitaxel solution, the preservative, the mineral, the fragrance essential oil and the water according to a formulation ratio;
S2, heating the oil to 70° C. to 85° C., and adding the auxiliary emulsifier into the oil, followed by stirring to disperse, thereby obtaining an oil phrase;
S3, heating the water to above 95° C., and adding the humectant into the water, followed by stirring evenly to obtain a mixture; and adding the emulsifier into the mixture, followed by stirring to disperse, thereby obtaining an emulsion;
S4, controlling a temperature of the emulsion obtained in step S3 to 70° C. to 85° C., and slowly injecting the oil phrase obtained in step S2 into the emulsion, followed by stirring evenly to obtain a matrix; and
S5, slowly cooling the matrix obtained in step S4 to 45° C. to 55° C., and sequentially adding the paclitaxel solution, the preservative, the fragrance essential oil and the mineral into the matrix, followed by stirring to disperse evenly and cooling to below 40° C. to obtain the depilatory emulsion containing paclitaxel.
